(19) United States
(12) Patent Application Publication
(10) Pub. No.: US 2013/0144256 A1
(43) Pub. Date: Jun. 6, 2013

(54) MEDICAL MARKING APPARATUS AND METHODS

(71) Applicants: Paul Howard Wessel, Delano, MN (US); Rachel Jacques, Plymouth, MN (US)

(72) Inventors: Paul Howard Wessel, Delano, MN (US); Rachel Jacques, Plymouth, MN (US)

(21) Appl. No.: 13/486,639

(22) Filed: Jun. 1, 2012

Related U.S. Application Data
(60) Provisional application No. 61/492,226, filed on Jun. 1, 2011.

(51) Int. Cl. A61M 5/42

(52) U.S. Cl. CPC A61M 5/427
USPC 604/506; 604/116

(57) ABSTRACT

The present invention provides the materials and methods related to marking the injection site on a patient's skin. Specifically, the present invention provides the materials and methods for applying a medical marking tattoo containing at least one injecting targeting site, injecting a medical substance into the area of the injection target site, and marking the injecting targeting site using a tattoo activation element. The present invention also provides the materials and methods for a medical marking apparatus that can be used to perform the methods of the present invention to mark the injection site on a patient's skin.

### RELATED APPLICATION

The present application claims the benefit of U.S. Provisional Application No. 61/492,226 filed Jun. 1, 2011, which is incorporated herein in its entirety by reference.

### TECHNICAL FIELD

This disclosure generally relates to the field of medical marking devices. This disclosure also relates to the application of a body treating material on the outer surface of the body where the injector is entirely supported during application or injection.

### BACKGROUND

According to the U.S. Department of Health and Human Services, Centers for Disease Control and Prevention, about 215,000 people aged 20 or younger have diabetes (Type I or Type 2). Between the years 2002-2005, 15,600 youths were diagnosed with Type 1 diabetes annually, and the rate of new cases for children aged 10 years or younger was 19.7 per 100,000 each year. Type I diabetes typically strikes children and young adults but the disease can occur at any age.

Type I diabetes (known as diabetes mellitus or juvenile-onset diabetes) develops when the body's immune system attacks and destroys the pancreatic beta cells. The beta cells are the only cells that produce the hormone insulin, which regulates glucose. Insulin helps the body transport the glucose contained in foods to cells throughout the body, where it is subsequently used for energy or stored. However, when beta cells are destroyed, the glucose remains in the blood, which leads to insulin resistance and serious organ damage.

Currently, there is no way to prevent Type I diabetes, and to survive, people must have insulin delivered by injection or pump. Diabetes management is complex and overwhelming, especially for parents (caregivers) and their newly diagnosed child. On a daily basis the caregivers must help their child count carbohydrates, test their blood, monitor exercise and inject or pump insulin into their bodies. Regular rotation of insulin sites is a standard recommendation for diabetes self-care. It is important that the injection site be regularly rotated to prevent lipodystrophy and promote better absorption.

Failure to follow proper injection site rotation, for any reason, can have severe long-term consequences on the wellness of the diabetic adolescent and contribute to significant increases in future health care costs. Lipodystrophy is the most common adverse consequence of inadequate insulin site rotation. Lipodystrophy is a degenerative disorder of the subcutaneous tissue that can cause thickening of the tissue and lumps or dents in the affected areas. It can reduce absorption of insulin at the injection site by as much as 25%, leading to inferior glucose control. In some patients, lipodystrophy results in higher total doses of insulin being injected into an individual, in order to achieve optimal glycemic control. Despite this severe health risk, a recent study concluded that many youths fail to adhere to an adequate site rotation plan (Survey of Insulin Site Rotation in Youth with Type 1 Diabetes Mellitus). Some of the reasons given for not adequately rotating the injection site involve comfort with the existing routine and concern with reaching unfamiliar injection sites.

Adequate injection site rotation is also important for controlling the symptoms of Multiple Sclerosis (MS), an autoimmune disease. MS is a disease in which the immune system attacks the brain and spinal cord. Scar tissue (sclerosis) or plaques form in the brain and/or spinal cord when, myelin, the protective sheath covering nerve fibers, are destroyed. Without this myelin, the electrical signals transmitted throughout the brain and spinal cord deteriorate, and the brain is unable to send and receive signals. The symptoms of MS are a result of the breakdown in this transmission.

The symptoms of MS vary from person-to-person and may include abnormal fatigue, tingling/pain, changes in vision, loss of balance and muscle coordination, depression/emotional changes, numbness, slurred speech, tremors, muscle spasticity, bladder and/or bowel problems, and in severe cases, partial or complete paralysis. The National Multiple Sclerosis Society estimates that there are approximately 300,000 to 400,000 people in the United States with MS, with approximately 200 people diagnosed each week. MS is two to three times as common in women as in men, with the first symptoms usually occurring between the ages of 20 and 40. No cure currently exists.

However, there are several drugs available that have been shown to slow the progression of MS and reduce the frequency and severity of MS attacks. Interferon beta-1a is used for the treatment of relapsing forms of MS and for treatment after an initial episode of MS. Interferon beta-1a is typically injected weekly into the muscle. Interferon beta-1b is also used for the treatment of relapsing forms of MS and is subcutaneously injected every other day. Due to the frequency of injection, most brand name interferon beta-1b drugs advise patients of the importance of injection site rotation and in some studies, injection site necrosis has been reported in 4% of patients in controlled trials (BETASERON® Safety Information 2011, http://www.betaseron.com/safety.jsp). Accordingly, there exists a need for tools to help patients and their caregivers implement adequate injection site rotation in chronic diseases such as Type I diabetes and MS. The present disclosure of medical marking devices and methods, as described below, addresses this limitation.

### SUMMARY

In general, failure to properly rotate the injection site can have severe long-term consequences on patient wellness and can significantly increase health care costs. Adequate injection site rotation is important for controlling the symptoms of diseases that require multiple injections during treatment, including Multiple Sclerosis (MS), diabetes, and other autoimmune diseases. A method for ensuring patient compliance with a protocol of adequate injection site rotation is therefore desirable.

The present invention relates generally to the materials and methods involved in marking an injection site on a patient's skin. Specifically, the present invention features the materials and methods for applying a medical marking tattoo containing at least one injection targeting site, injecting a medical substance into the area of the injection target site, and marking the injection targeting site using a tattoo activation element. The present invention also provides the materials and methods for a medical marking apparatus that can be used to perform the methods of the present invention to mark the injection site on a patient's skin.

In one aspect, the method of the present invention includes the application of a temporary medical marking tattoo that can contain between about 1 and about 14 different injection target sites. For example, the medical marking tattoo can contain about 1, 2, 3, 4, 5, 6, 7, 8, 9, 10, 11, 12, 13 or 14 injection targeting sites. In some embodiments, the medical marking tattoo comprises a general design of an ornament, symbol, cartoon, popular fictional character, or a licensed trademark that is pleasing to children and young adults (e.g., FIG. 2). In some aspects, the medical marking tattoo can be a temporary skin tattoo made up of several layers of material interspersed with dye or ink-permeable layers, which can be visible on the patient's skin for a period of at least two weeks.

Once the medical marking tattoo has been applied, a patient, caregiver or other qualified professional can use an injection device to inject a medical substance at the injection target site delineated within the medical marking tattoo. In some aspects, a patient, caregiver or other qualified professional can inject a medical substance to treat diabetes, Multiple Sclerosis or other autoimmune disease that requires periodic injections for treatment. For example, a patient, caregiver or other qualified professional can inject insulin, interferon beta-1a or interferon beta-1b. In some aspects, the medical substance injected by the injection device can be a nutritive substance designed to provide sustenance to a patient.

In some aspects, the medical marking apparatus can consist of an injection device bonded (e.g., thermally or mechanically) to a tattoo activation element, wherein the tattoo activation element is used to mark the injection target site before, after, or simultaneously with the injection of the medical substance (e.g., FIG. 3). In some aspects, the medical marking apparatus can consist only of a tattoo activation element, and the injection device can be used separately (e.g., FIG. 4). The tattoo activation element can mark the area around the injection target site using a needle and syringe, a piston pump or a plunger and barrel, all of which can be used to deliver medical ink or dye to the area of the injecting targeting site. In this way, the tattoo activation element provides visual confirmation of where the injection occurred and can ensure that the injection target site is properly rotated for the next dose of medication. In some aspects, the medical ink or dye used to mark the injection targeting site can be contained within a protective cap on the tattoo activation element (e.g., FIG. 7). The tattoo activation element can also be used to deliver medical dye or ink that can be magnetically coupled to a medical marking tattoo. In some aspects, the medical marking apparatus can further comprise an infusion pump, an infusion catheter, an insertable needle assembly, or an adhesive patch (e.g., FIG. 8). Once a medical substance has been injected and the injection site is marked, the method of the present invention can be repeated, or a new medical marking tattoo can be applied to another area of the patient's skin and the process can begin again.

### BRIEF DESCRIPTION OF THE DRAWINGS

FIG. 1 is a flow chart illustrating an exemplary embodiment of a method for applying a medical marking tattoo on the body and injecting a medical substance at an injection target site indicated on the tattoo.

FIG. 2 is a schematic illustration of an exemplary embodiment of a medical marking tattoo according to the present disclosure.

FIG. 3 is a schematic illustration of an exemplary medical marking apparatus according to the present disclosure.

FIG. 4 is a schematic illustration of an exemplary embodiment of a tattoo activation element according to the present disclosure.

FIG. 5 is an alternative medical marking apparatus according to the present disclosure.

FIG. 6 is a flow chart illustrating an exemplary alternative embodiment of a method for a medical marking apparatus tattoo.

FIG. 7 is a schematic illustration of an exemplary alternative medical marking apparatus according to the present disclosure.

FIG. 8 is a schematic illustration of an exemplary alternative medical marking apparatus according to the present disclosure.

FIG. 9 is a schematic illustration of an exemplary embodiment of the medical marking apparatus used on a patient's body.

### DETAILED DESCRIPTION OF THE INVENTION

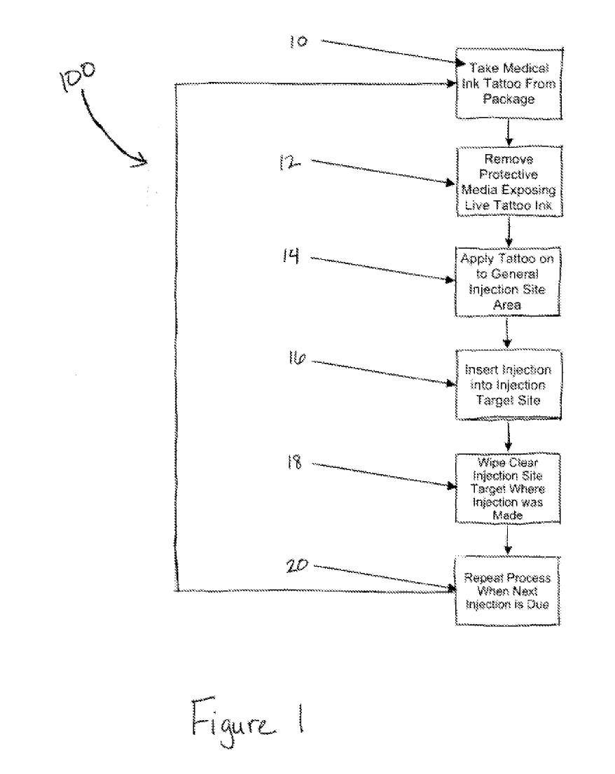

FIG. 1 illustrates an exemplary embodiment of a method of applying a medical ink tattoo and injecting a medical substance at the injection site. This method (100) includes removing the medical marking ink tattoo from the manufacturer's packaging (10); removing the protective media and exposing live tattoo ink (12); applying the tattoo to the general injection site area (14); inserting the injection device at the injection target site (16); wiping the injection target site with an antiseptic after injection of the medical substance (18); and then repeating the process when medically necessary (20).

The term ‘medical marking tattoo’ is used to describe any temporary surface treatment applied to the skin of an individual. Thus, ‘medical marking tattoo’ technology may include several layers of FDA approved materials interspersed with dye or ink-permeable and dye or ink-impermeable layers, so the individual's skin is exposed to the dye or ink and a tattoo will form from the layers permeable to the dye or ink. The individual may cut out and remove a pattern from the layers that transfers onto the skin when dye or ink is applied on one side.

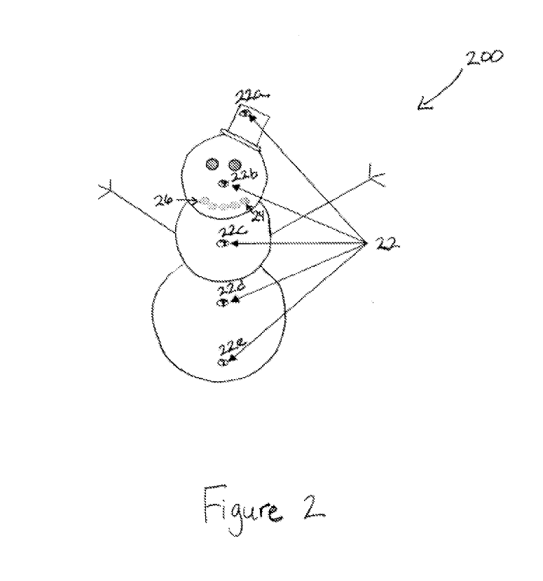

Referring to FIG. 2, exemplary medical marking tattoo (200) includes easily identifiable target sites (22a, 22b, 22c, 22d, and 22e). The medical marking tattoo is a temporary skin tattoo in the general design of an ornament, symbol, cartoon, popular fictional character, or a licensed trademark that is pleasing to children and young adults. Exemplary medical marking tattoo (200) is configured into the shape of a snowman and has five injection target sites (22a, 22b, 22c, 22d, and 22e). In one embodiment, two additional injection target sites may be included so that a medical marking tattoo may be used for up to one week. In another embodiment, a medical marking tattoo may contain enough injection target sites to last two weeks.

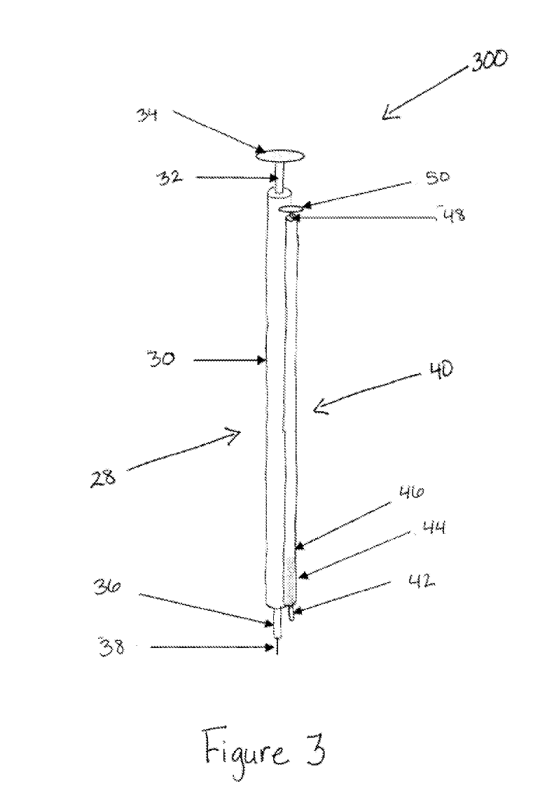

Referring to FIG. 3, an exemplary medical marking apparatus (300) includes a syringe (28) containing medicine (e.g., insulin) and is bonded to a tattoo activation element (40). In one embodiment, the syringe is a piston pump which includes a plunger (32) that extends throughout the length of the barrel (30) of the syringe in order to expel liquid or gas from the fluid reservoir. Located at one end (proximal) of the plunger is a flat surface perpendicular to a plunger, called a thumb or finger pad (34). Pressure is applied to the thumb or finger pad in order to expel liquid or gas from a syringe. A tensile force may also be applied to the thumb or finger pad in order to draw in liquid or air. At the other end (distal) of a syringe, there may be a needle or other injection device (38) which punctures the skin at the injection target site and delivers a medical substance (e.g., insulin). A needle can range from 5 to 330 mm in length, and from 25 to 33-gauge in thickness, or any other reasonable size depending on the individual patient. Finally, the barrel of a syringe may taper to a needle by way of a fitting (36). Such a fitting may be of a luer lok® design, a slip fit design, a catheter tip design, an eccentric tip design, or any other design suited to the individual patient.

FIG. 3 also refers to a tattoo activation element (40), which is coaxially aligned with the axis of tan injection device using a bonding process. The bonding process may be thermal or mechanical. A tattoo activation element functions similarly to a syringe. It may include a plunger (48) that extends throughout the barrel (46) of a tattoo activation element in order to expel liquid or gas from the fluid reservoir. Located at one of (proximal) end of the plunger is a flat surface perpendicular to a plunger called a thumb or finger pad (50). Pressure is applied to a thumb or finger pad in order to expel liquid or gas from the tattoo activation element. A tensile force may also be applied to the thumb or finger pad 50 in order to draw in liquid or air. At the other end (distal) of the tattoo activation element, there is a needle (42) through which medical ink (44) is delivered to the skin at, or in close proximity to, the injection target site where a medical substance was injected or applied (e.g., insulin). A needle can range from 5 to 330 mm in length, and from 25 to 33-gauge in thickness, although the size may vary according to the individual patient. Alternatively, the tattoo activation element can be contained within a syringe.

The medical ink or dye (44) is preferably FDA-approved. In one embodiment, medical ink or dye is of a different pigmentation than the medical marking tattoo (200) of FIG. 2. In another embodiment, the medical ink or dye, after application, is in the general design of an ornament, symbol, cartoon, popular fictional character, or a licensed trademark that is pleasing to children and young adults. In another embodiment, an antiseptic agent is delivered prior to, or after, the medical ink or dye is applied so that the injection target sites are sterilized. In another embodiment, medical ink or dye is magnetically coupled to the medical marking tattoo (200) of FIG. 2. After the skin is marked with medical ink or dye, the patient and his or her caregiver has visual confirmation of where the injection occurred and can ensure that the injection target site is properly rotated for the next dose of medication. At this time, the method of applying a medical marking tattoo may be repeated.

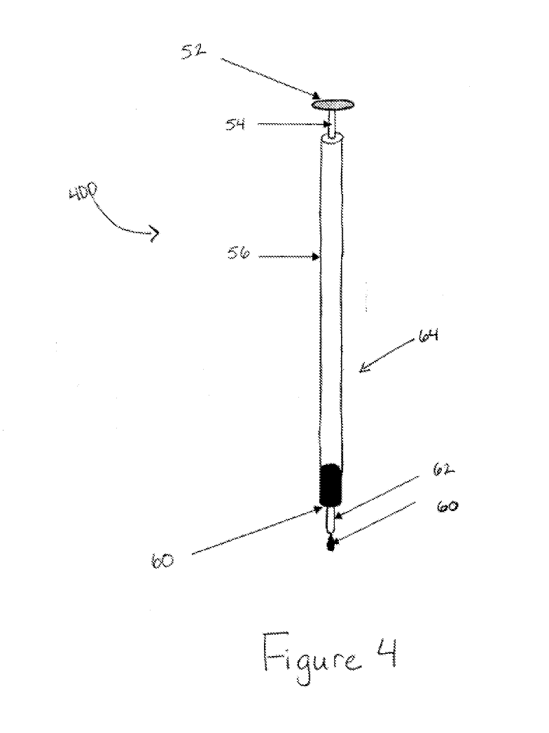

Referring to FIG. 4, an exemplary medical marking apparatus (400) includes a tattoo activation element (64) that expels medical ink or dye (60), without being coupled to a syringe that injects medication. A tattoo activation element may also include a plunger (54) that extends throughout the barrel (56) of the tattoo activation element in order to expel liquid or gas. Located at one of (proximal) end of the plunger is a flat surface, perpendicular to the plunger, called a thumb or finger pad (52). Pressure is applied to the thumb or finger pad in order to expel liquid or gas from tattoo activation element. A tensile force may also be applied to the thumb or finger pad in order to draw in liquid or air. At the other end (distal) of tattoo activation element there is a needle (62), through which medical ink or dye is delivered to the skin at, or in close proximity to, the injection target site where a medical substance was injected or applied (e.g., insulin). A needle can range from 5 to 330 mm in length, and from 25 to 33-gauge in thickness, although the size may vary depending on the individual patient.

The medical ink or dye is preferably FDA-approved. In one embodiment, medical ink or dye is of a different pigmentation than the medical marking tattoo (200) of FIG. 2. In another embodiment, medical ink or dye, after application, is in the general design of an ornament, symbol, cartoon, popular fictional character, or a licensed trademark that is pleasing to children and young adults. In another embodiment, an antiseptic agent is delivered prior to, or after, the medical ink or dye is applied so that the injection target sites are sterilized. In another embodiment, medical ink or dye is magnetically coupled to the medical marking tattoo (200) of FIG. 2. After the skin is marked with medical ink or dye, the patient and his or her caregiver has visual confirmation of where the injection occurred and can ensure that the injection target site is properly rotated for the next dose of medication. At this time, the method of applying a medical marking tattoo may be repeated.

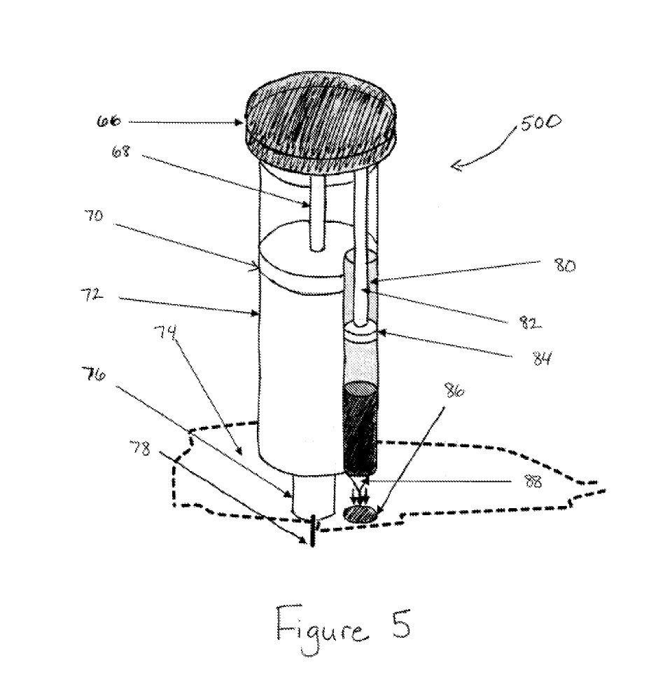

Referring to FIG. 5, a medical marking apparatus (500) is depicted that includes a syringe (70) operably connected to a tattoo activation element (84) by a primary plunger (68). The syringe is a piston pump that includes a primary plunger that extends throughout the barrel (72) of the syringe in order to expel liquid or gas. Located at one of end (proximal) of the primary plunger is a flat surface perpendicular to primary plunger called a thumb or finger pad. Pressure is applied to the thumb or finger pad in order to expel liquid or gas from the syringe. A tensile force may also be applied to the thumb or finger pad in order to draw in liquid or air. At the other end (distal) of the syringe, 70 there may be a needle or other injection device (78) which punctures the skin at the injection target site (74) and delivers a medical substance (e.g., insulin). The needle can range from 5 to 330 mm in length, and from 25 to 33-gauge in thickness, or any other reasonable size depending on the individual patient. Finally, the barrel (72) of the syringe may taper to a needle by way of a fitting (76). The fitting may be of a luer lok® design, a slip fit design, a catheter tip design, an eccentric tip design, or any other design suited to the individual patient.

The tattoo activation element is coaxially aligned with the axis of an injection device by way of a bonding process. The bonding process may be thermal or mechanical. A tattoo activation element may have a secondary plunger (80 and 82) connected to the thumb or finger pad of a syringe, such that medical ink or dye (86) is not distributed from a needle (88) onto the target injection site until the primary plunger has traveled the length of the barrel.

The medical ink or dye is preferably FDA-approved. In one embodiment, the medical ink or dye is of a different pigmentation than the medical marking tattoo (200) of FIG. 2. In another embodiment, the medical ink or dye, after application, is in the general design of an ornament, symbol, cartoon, popular fictional character, or a licensed trademark that is pleasing to children and young adults. In another embodiment, an antiseptic agent is delivered prior to, or after, the medical ink or dye 86 so the injection target sites are sterilized. In another embodiment, the medical ink or dye is magnetically coupled to the medical marking tattoo (200) of FIG. 2. After the skin is marked with medical ink or dye, the patient and his or her caregiver has visual confirmation of where the injection occurred and can ensure that the injection target site is properly rotated for the next dose of medication. At this time, the method of applying a medical marking tattoo may be repeated.

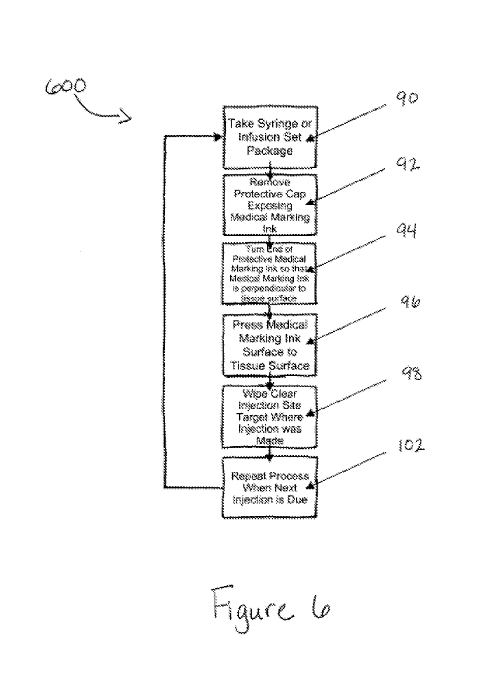

FIG. 6 illustrates an alternative embodiment of a method (600) for applying a medical marking at an injection site. This method includes procuring the medical marking kit from the manufacturer (90); removing the protective cap from the syringe (92); positioning the cap opening perpendicular to the surface of the skin (94); pressing the cap downward (96); wiping the injection target site with an antiseptic (98); and repeating the process at the next injection target site as medically needed (102).

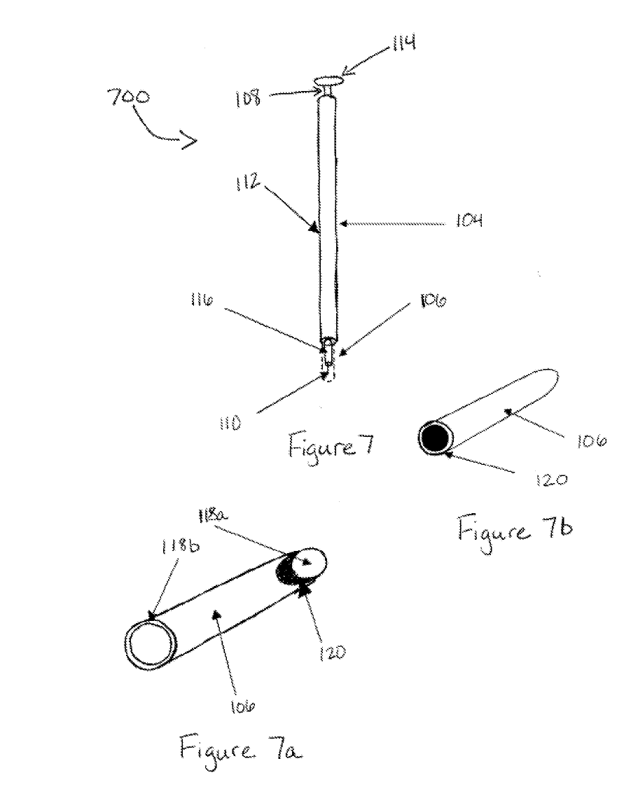

Referring to FIG. 7, a medical marking apparatus (700) may include a syringe (104) with protective cap (106). A syringe may be a piston pump which includes a plunger (108) that extends throughout the barrel (112) of the syringe in order to expel liquid or gas. Located at one of end (proximal) of the plunger is a flat surface perpendicular to the plunger called a thumb or finger pad (114). Pressure is applied to the thumb or finger pad in order to expel liquid or gas from the syringe. A tensile force may also be applied to the thumb or finger pad in order to draw in liquid or air. At the other end (distal) of the syringe there may be a needle (110) which punctures the skin at the injection target site and delivers a medical substance (e.g., insulin). A needle can range from 5 to 330 mm in length, and from 25 to 33-gauge in thickness, or any other reasonable size depending on the individual patient. Finally, the barrel of the syringe may taper to a needle by way of a fitting (116). The fitting may be of a luer lok® design, a slip fit design, a catheter tip design, an eccentric tip design, or any other design suited to the individual patient.

Referring to FIG. 7a, the protective cap of a medical marking apparatus (700) may include medical ink or dye (120) encompassed within the closed end (118a) of the protective cap. After a medical substance has been delivered to the injection target site with the needle, the open end (118b) of the protective cap is positioned perpendicular to the skin's surface and force is applied. The force displaces the medical ink or dye to the opening of the protective cap, as shown in FIG. 7b, and medical ink or dye is applied to the surface of the skin. After the skin is marked with medical ink or dye, the patient and his or her caregiver has visual confirmation of where the injection occurred and can ensure that the injection target site is properly rotated for the next dose of medication. At this time, use of medical marking apparatus may be repeated.

The medical ink or dye is preferably FDA-approved. In one embodiment, the medical ink or dye is of a different pigmentation than the medical marking tattoo (200) of FIG. 2. In another embodiment, the medical ink or dye, after application, is in the general design of an ornament, symbol, cartoon, popular fictional character, or a licensed trademark that is pleasing to children and young adults. In another embodiment, an antiseptic agent is delivered prior to, or after, medical ink or dye so that the injection target sites are sterilized. In another embodiment, the medical ink or dye is magnetically coupled to the medical marking tattoo (200) of FIG. 2.

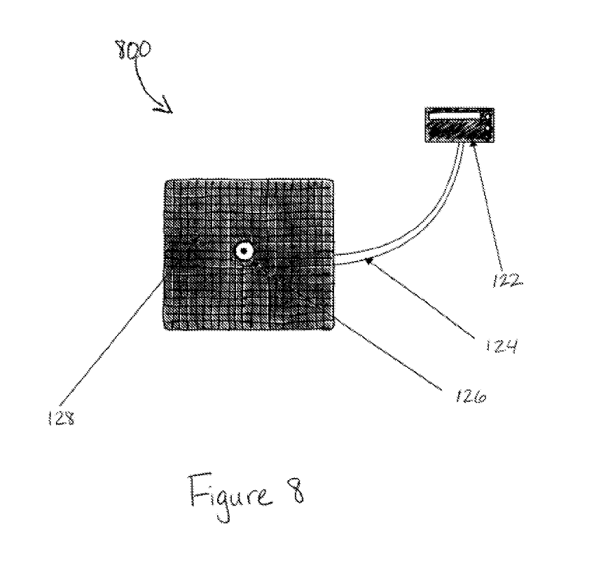

Referring to FIG. 8, a medical marking apparatus (800) may include an infusion pump (122), an infusion catheter (124), an insertable needle assembly (126), of an adhesive patch (128). An adhesive patch may contain medical ink or dye. The medical marking apparatus can be adapted so medical ink or dye is deposited onto the surface of the skin after the adhesive patch has been positioned. Alternatively, an adhesive patch of a medical marking apparatus can also contain an antiseptic agent working simultaneously with the medical ink or dye.

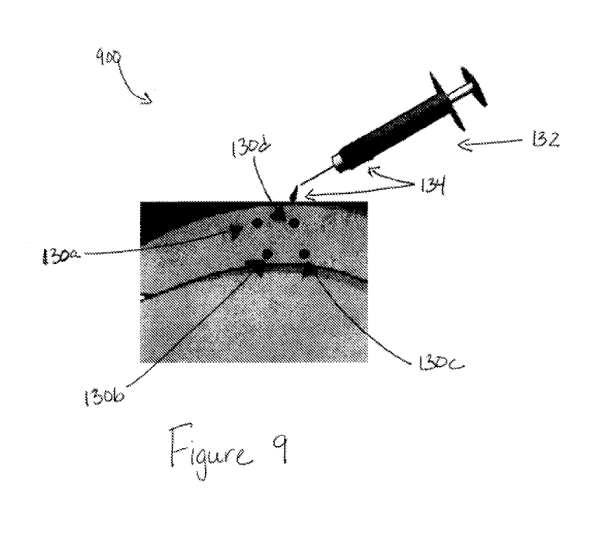

Referring to FIG. 9, the general injection site (900) may include easily identifiable target sites (130a, 130b, 130c, and 130d). A tattoo activation element (132) may contain a medical ink or dye (134) which is a different pigmentation than the tattoo left on a target site After the appropriate medical injection has been made into target sites, the patient is left with medical ink or dye on the surface of his or her skin for up to two weeks. After the skin is marked with medical ink or dye, the patient and his or her caregiver has visual confirmation of where the injection occurred and can ensure that the injection target site is properly rotated for the next dose of medication.

Various modifications and alterations of this invention will become apparent to those skilled in the art without departing from the scope and spirit of this disclosure, and it should be understood that this invention is not to be unduly limited to the illustrative embodiments set forth herein.

What is claimed is:

1. A method for temporarily marking an injection site on a patient's skin comprising the steps of:
applying a medical marking tattoo containing at least one injection target site to a patient's skin where an injection is to be made,
injecting a medical substance into said injection target site contained within said medical marking tattoo using an injection device, and
marking an area of skin near said injection target site and within said medical marking tattoo using a tattoo activation element to indicate that an injection has been delivered at the injection target site.

2. The method of claim 1, wherein said medical marking tattoo comprises between 1 and 14 injection target sites.

3. The method of claim 1, wherein said medical marking tattoo comprises a temporary skin tattoo comprising several layers of materials interspersed with dye or ink-impermeable layers.

4. The method of claim 1, wherein said medical substance is a substance used to treat diabetes, multiple sclerosis or another autoimmune disease.

5. The method of claim 1, wherein said medical substance is insulin, interferon beta-1a or interferon beta-1b.

6. A medical marking apparatus used for marking an injection site on a patient's skin comprising:
a tattoo activation element that is attached to and coaxially aligned with an injection device, wherein said tattoo activation element comprises a fluid reservoir with a needle connected to its distal end and a plunging device, said needle being capable of delivering medical ink or dye to a patient's skin when force is applied to the plunging device, and
wherein said injection device coaxially aligned with the axis of the tattoo activation element comprises a fluid reservoir with a needle connected to its distal end and a plunging device, said needle being capable of delivering medical or nutritive substances to a patient's skin when force is applied to the plunging device.

7. The medical marking apparatus of claim 6, wherein said fluid reservoir of said tattoo activation element contains medical ink or dye.

8. The medical marking apparatus of claim 6, wherein said fluid reservoir of said tattoo activation element is a protective cap, said medical ink or dye being capable of being displaced to a patient's skin when force is applied to said protective cap.

9. The medical marking apparatus of claim 6, wherein said fluid reservoir of said tattoo activation element contains medical dye or ink that can be magnetically coupled to a medical marking tattoo.

10. The medical marking apparatus of claim 6, wherein said plunging device of said tattoo activation element further comprises a syringe containing medical ink or dye connected to a needle, said medical ink or dye being capable of being displaced onto patient's skin when force is applied to said syringe.

11. The medical marking apparatus of claim 6, wherein said plunging device of said tattoo activation element further comprises a piston pump and a plunger containing medical ink or dye connected to a needle, said medical ink or dye being capable of being displaced onto patient's skin when force is applied to said piston pump.

12. The medical marking apparatus of claim 6, wherein said plunging device of said tattoo activation element further comprises a barrel and plunger containing medical ink or dye connected to a needle, said medical ink or dye being capable of being displaced onto patient's skin when force is applied to said plunger.

13. The needle and plunging device of claim 10, wherein said needle is connected to said plunging device using a luer lok design, a slip fit design, a catheter tip design, or an eccentric tip design.

14. The medical marking apparatus of claim 6, wherein the medical marking apparatus further comprises an infusion pump, an infusion catheter, an insertable needle assembly, or an adhesive patch.
